(19) United States
(12) Patent Application Publication
(10) Pub. No.: US 2025/0161628 A1
(43) Pub. Date: May 22, 2025

(54) PACKAGING ARRANGEMENT FOR A MEDICAL DEVICE AND CONNECTING ELEMENT FOR SUCH A PACKAGING ARRANGEMENT

(71) Applicant: BIOTRONIK AG, Bülach (CH)

(72) Inventors: Moritz Oeler, Zuerich (CH); Johannes Schampel, Birmensdorf (CH); Christopher Lenherr, Winterthur (CH); Markus Hepke, Waltrop (DE)

(21) Appl. No.: 18/833,308

(22) PCT Filed: Jan. 13, 2023
(86) PCT No.: PCT/EP2023/050689

§ 371 Date: Jul. 25, 2024

(30) Foreign Application Priority Data
Feb. 15, 2022 (EP) 22156697.9

(51) Int. Cl. A61M 25/00; B65D 21/02

(52) U.S. Cl. CPC A61M 25/002; B65D 21/0204

(57) ABSTRACT

A detachable connecting element for a packaging arrangement includes a first connecting element part and a second connecting element part configured for relative movement with respect to each other around a connecting element axis (R) by a rotational or helical movement to transfer the detachable connecting element from a linking state to a non-linking state. The second connecting element part includes at least one protrusion. The first connecting element part includes at least one recess configured to incorporate the at least one protrusion. The at least one protrusion includes at least one curved protrusion sidewall. The at least one recess includes at least one curved recess sidewall. The at least one curved protrusion sidewall or the at least one curved recess sidewall includes a slope.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2023/050689, which was filed Jan. 13, 2023, which application claimed priority from EP Application Serial No. 22156697.9, which was filed Feb. 15, 2022.

### FIELD OF THE INVENTION

A field of the invention concerns packaging arrangements, and particularly packaging arrangements for medical devices.

### BACKGROUND

US 2012/0305441 A1 discloses a packaging for a delivering catheter system. The packaging includes two packaging parts that are connected to each other with a hinge. This hinge enables a relative movement between the two packaging parts; however, it keeps those packaging parts always closely together.

Such an arrangement has certain deficiencies. On the one hand, it requires a sophisticated design of the packaging parts in order to move them relative to each other around the hinge. On the other hand, the overall possibilities of arranging the packaging parts with respect to each other are limited, e.g. as to their free movement in relation to one another.

### SUMMARY OF THE INVENTION

A detachable connecting element for a packaging arrangement includes a first connecting element part and a second connecting element part configured for relative movement with respect to each other around a connecting element axis (R) by a rotational or helical movement to transfer the detachable connecting element from a linking state to a non-linking state. The second connecting element part includes at least one protrusion. The first connecting element part includes at least one recess configured to incorporate the at least one protrusion. The at least one protrusion includes at least one curved protrusion sidewall. The at least one recess includes at least one curved recess sidewall. The at least one curved protrusion sidewall or the at least one curved recess sidewall includes a slope.

A packaging arrangement for a medical device includes the detachable connecting element, a first packaging part, and a second packaging part arranged with the detachable connecting element to be connectable into a connecting state. The packaging arrangement can include at least one detachable connecting rinsing tray configured to store and/or receive a liquid. The at least one detachable connecting rinsing tray is preferably a one-piece element that contacts the first packaging part and the second packaging part in the connecting state and is configured to be removed from the at least one of the first packaging part and the second packing part in a non-connecting state.

### BRIEF DESCRIPTION OF THE DRAWINGS

Further details of aspects of the present invention will be explained with respect to exemplary embodiments and accompanying Figures. In the Figures:

FIG. 1 shows a first embodiment of a packaging arrangement for a medical device in a first state;

FIG. 2 shows the packaging arrangement of FIG. 1 in a second state;

FIG. 3 shows a schematic depiction of the packaging arrangement of FIG. 1 in a third state;

FIG. 4A shows a second embodiment of a packaging arrangement for a medical device in a first state;

FIG. 4B shows the packaging arrangement of FIG. 4A in a second state;

FIG. 4C shows the packaging arrangement of FIG. 4A in a third state;

FIG. 5A is a first detail view of a connecting area of the packaging arrangement of FIG. 4A;

FIG. 5B is a second detail view of the connecting area of the packaging arrangement of FIG. 4A;

FIG. 6A shows a first embodiment of a connecting element for a packaging arrangement with two engaged packaging parts;

FIG. 6B shows the connecting element of FIG. 6A with disengaged packaging parts;

FIG. 7A shows a second embodiment of a connecting element for a packaging arrangement with two engaged packaging parts; and

FIG. 7B shows the connecting element of FIG. 7A with disengaged packaging parts.

### DETAILED DESCRIPTION OF THE PREFERRED EMBODIMENTS

A preferred detachable connecting element includes a first connecting element part and a second connecting element part. The first connecting element part and the second connecting element part are relatively movable to each other around a connecting element axis by a rotational or helical movement. The detachable connecting element can be present in a linking state and in a non-linking state. In the non-linking state the first connecting element part and the second connecting element part are completely separated from each other. In the linking state the first connecting element part and the second connecting element part are attached/connected to each other. The first connecting element part may be connected to a first base body and the second connecting element part may be connected to a second base body. The connecting element axis is perpendicular to the first base body of the first connecting element part and to the second base body of the second connecting element part (when the first connecting element part and the second connecting element part are in the linking state). This movement around the connecting element axis leads to a transfer of the connecting element from its linking state to its non-linking state (and optionally vice versa). The second connecting element part includes at least one protrusion and the first connecting element part includes at least one recess for incorporating one of the at least one protrusions of the second connecting element part. At least one of the protrusions of the second connecting element part has at least one curved protrusion sidewall and at least one of the recesses of the first connecting element part has at least one curved recess sidewall. The at least one curved recess sidewall or at least one recess sidewall of the at least one recess has a slope (pointing away from the first base body).

The at least one curved protrusion sidewall or at least one protrusion sidewall of the at least one protrusion may have a slope (pointing away from the second base body). At least one of the protrusions may be a curved and/or bent protrusion having at least one protrusion sidewall. In the linking state the at least one protrusion sidewall may have a slope towards the at least one recess of the first connecting element part.

In another embodiment at least one of the protrusion may be a curved and/or bent protrusion having at least two protrusion sidewalls, wherein at least two protrusion sidewalls being adjacent to each other and wherein at least one protrusion sidewall has a slope towards the at least one corresponding recess of the first connecting element part (in the linking state).

The first connecting element part includes at least one recess protruding from a base body of the first connecting element part when looking along the connecting element axis in one direction and the second connecting element part includes at least one protrusion protruding from a base body of the second connecting element part when looking along the connecting element axis in the same direction (when referred to the linking state). The recess of the first connecting element part can be connected to the protrusion of the second connecting element part by a form fit and/or force fit connection.

At least one of the protrusions, preferably two protrusions, of the second connecting element part can have x protrusion sidewalls with x being 2 to 6, y edges with y being 6 to 12 and z vertices with z being 5 to 8. At least one of the protrusion sidewalls has n adjacent protrusion sidewalls with n being 1 to 5.

One of the at least one protrusion may be limited by at least two to six protrusion sidewalls. The protrusion can be defined/limited by at least two, three or four protrusion sidewalls, with at least two adjacent protrusion sidewalls (meaning two protrusion sidewalls being adjacent to each other or one protrusion sidewall has one adjacent protrusion sidewall). At least two protrusion sidewalls, preferably three or four protrusion sidewalls, are attached to the base body of the second connecting element part.

At least one of the recesses, preferably two recesses, of the first connecting element part can have x recess sidewalls with x being 2 to 6, y edges with y being 6 to 12 and z vertices with z being 5 to 8. At least one of the recess sidewalls has n adjacent recess sidewalls with n being 1 to 5. One of the at least one recesses may be limited by at least two to six recess sidewalls. The recess can be defined/limited by at least two, three or four recess sidewalls, with at least two adjacent recess sidewalls (meaning two recess sidewalls being adjacent to each other or one recess sidewall has one adjacent recess sidewall). At least two recess sidewalls, preferably three or four recess sidewalls, are attached to the base body of the first connecting element part.

Table 1 shows specific embodiments of protrusion and corresponding recess geometries. According to Table 1 at least one of the protrusions and/or at least one of the recesses, preferably two protrusions and/or two recesses, can have x protrusion or recess sidewalls with y edges and z vertices, having at least one protrusion and/or recess side wall with n adjacent protrusion or recess sidewalls.

| | one sidewall | | |
|---|---|---|---|
| | having n adjacent | | |
| x sidewalls | sidewalls | y edges | z vertices |
| 2 | 1 | 6 | 5 |
| 2 | 1 | 6 | 5 |
| 2 | 1 | 7 | 6 |
| 3 | 2 | 8 | 6 |
| 3 | 1 | 8 | 6 |
| 3 | 2 | 7 | 6 |
| 4 | 2 | 9 | 6 |
| 4 | 2 | 10 | 7 |
| 4 | 3 | 8 | 6 |
| 5 | 4 | 11 | 7 |
| 6 | 5 | 12 | 8 |

In one embodiment the recess and the protrusion each can have two recess or protrusion sidewalls being adjacent to each other (in other words one recess or protrusion sidewall has one adjacent recess or protrusion sidewall), with six edges and five or six vertices.

In another embodiment the recess and the protrusion each can have three recess or protrusion sidewalls with eight edges and six vertices, with either two or three recess or protrusion adjacent sidewalls.

In a further embodiment the recess and the protrusion each can have four recess or protrusion sidewalls with six vertices and nine edges, wherein three recess or protrusion sidewalls being adjacent to each other (in other words one recess or protrusion sidewall has two adjacent recess or protrusion sidewalls). Such a recess can be referred to as ramp or wedge.

The at least one protrusion sidewall can have a slope towards the at least one (form fit) recess. The slope has angle of inclination. The angle of inclination is the angle at which the protrusion sidewall of the second connecting element parts rises towards the first connecting element (when the detachable connecting element being in the linking state).

At least one protrusion sidewall can have a slope. Preferably, the angle of inclination for at least one protrusion sidewall is less than 60 degrees and more than 1 degree. Preferably, the angle of inclination is between 10 degrees and 45 degrees or more preferably between 20 degrees and 30 degrees. The force needed for unlocking the detachable connecting element is higher for steeper angles of inclination. Thus, detachable connecting elements with protrusions having flatter angles (e.g. 1 to 20 degrees) are easier to unlock than recesses having steeper angles (e.g. more than 20 degrees). However, detachable connecting elements having steeper angles (e.g. more than 20 degrees) may require less rotation and less space. The slope may be a non-steady slope, e.g. a slope having a sigmoid function.

One of the at least one recesses and/or protrusions can have a bent triangular shaped recess and/or protrusion sidewall (a triangular shaped sidewall being bent around the rotation axis R).

At least one protrusion and/or recess can have a bent wedge shape. A wedge is a polyhedron defined by two triangles and three trapezoid faces. A wedge has five faces, nine edges, and six vertices. A bent wedge shape means that the protrusion and/or the recess has as basic shape a wedge shape (or ramp shape) which is bent (or curved/arcuated) around the rotation axis R.

At least one protrusion and/or recess can have the basic shape of a digonal cupola or triangular prism. A cupola is formed by joining two polygons, e.g. joining two wedges results in a digonal cupola. The basic shape of a digonal cupola or triangular prism can be bent around the rotation axis.

One of the at least one recesses and/or protrusions sidewall can have an indentation/and or protuberance (being oriented perpendicular to the rotation axis).

The first connecting element part can include at least one further recess and the second connecting element part can include at least one further protrusion. The at least one further protrusion can be a male connector and the at least one further recess can be a female connector. The male and female connector can act together as a press button, press stud, push button or snap fastener. The at least one female connector and the at least on male connector can a hemispherical shape or hemispherical like shape.

The detachable connecting element can be locked by rotating or twisting the first connecting element part or the second connecting element part in one direction around the rotation axis R and the detachable connecting element can be unlocked by rotating or twisting the first connecting element part or the second connecting element part in a direction opposite to the one direction. Thus, the detachable connecting element has a twist-lock and a twist-unlock function.

In one embodiment the detachable connecting element includes a first connecting element part and a second connecting element part, wherein the first connecting element part and the second connecting element part are relatively movable to each other around a connecting element axis by a rotational or helical movement to transfer the connecting element from a linking state to a non-linking state (and optionally vice versa). The second connecting element part includes two protrusions and the first connecting element part includes two recesses for incorporating the protrusions of the second connecting element part. The two protrusions of the second connecting element part each have at least one curved protrusion sidewall and the two recesses of the first connecting element part each have at least one curved recess sidewall. One of the protrusions and one of the recesses can have a bent wedge shape or the shape of a digonal cupola (fitting into each other). Alternatively, one of the recesses can have a bent wedge shape or the shape of a digonal cupola and one of the protrusions may be a pin (interacting with the bent shaped recess or the diagonal cupola shaped recess). The other protrusion can be a male connector and the other recess can be a female connector, preferably acting together as a press button, press stud, push button or snap fastener.

In another embodiment the detachable connecting element includes a first connecting element part and a second connecting element part, wherein the first connecting element part and the second connecting element part are relatively movable to each other around a connecting element axis by a rotational or helical movement to transfer the connecting element from a linking state to a non-linking state (and optionally vice versa). The second connecting element part includes three protrusions and the first connecting element part includes three recesses for incorporating the protrusions of the second connecting element part. The three protrusions of the second connecting element part each have at least one curved protrusion sidewall and the three recesses of the first connecting element part each have at least one curved recess sidewall. Two of the protrusions and two of the recesses can have a bent wedge shape or the shape of a digonal cupola (fitting into each other). Alternatively, two of the recesses can have a bent wedge shape or the shape of a digonal cupola and two of the protrusions may be a pin (interacting with the bent shaped recesses or the diagonal cupola shaped recesses). The other protrusion can be a male connector and the other recess can be a female connector, preferably acting together as a press button, press stud, push button or snap fastener.

It is another object of the present invention to provide a packaging, including the detachable connecting element. Such a packaging can be used for packaging any type of goods, for example a medical device, pharmaceuticals, food, consumer electronics or the like. Preferably, the packaging can be used for packaging a delivery catheter system, e.g. a TAVI/TAVR delivery catheter system; a heart valve prothesis, an aortic valve, a mitral valve, a pulmonal valve, a tricuspid valve, a (aortic) graft or a medical occluder like a left atrial appendage occluder, an atrial or ventricular septal defect occluder or a patent foramen ovale occluder. TAVI means Transcatheter Aortic Valve Implantation and TAVR means Transcatheter aortic valve replacement.

This object is achieved by a packaging arrangement having the features of claim 9.

A packaging arrangement, preferably for a medical device, is described and the packaging arrangement includes a first packaging part, a second packaging part and the aforementioned detachable connecting element for connecting the first packaging part and the second packaging part with each other. The detachable connecting element connects a portion of the first packaging part and a portion of the second packaging part in a connecting state and the detachable connecting element enables a relative movement between the first packaging part and the second packaging part in a non-connecting state and or the connecting state. The first connecting element part is arranged on or forms part of the first packaging part and the second connecting element part is arranged on or forms part of the second packaging part, or vice versa. The first connecting element part and the second connecting element part allow a relative movement of the first packaging part and the second packaging part around the connecting element axis. In this context, a movement of the first connecting element part is typically not possible without movement of the first packaging part (i.e., the first connecting element part is typically tightly connected to the first packaging part or forms an integral part of the first packaging part). Likewise, a movement of the second connecting element part is typically not possible without movement of the second packaging part (i.e., the second connecting element part is tightly connected to or forms an integral part of the second packaging part).

Such a packaging arrangement can be used for housing a medical device, in particular a medical device such as a delivery catheter system; e.g. a TAVI/TAVR delivery catheter system; a heart valve protheses or (leadless) pacemakers. The packaging arrangement includes an (oblong) first packaging part, an (oblong) second packaging part and a connecting element for connecting the first packaging part and the second packaging part with each other. In an oblong packaging part the length is the greatest dimension of the three dimensions (length, depth and height) of the packaging part. A packaging part can have a length of between 1 cm and 200 cm. A packing arrangement for a catheter e.g. a TAVI/TAVR delivery catheter can have a length of between 150 cm and 200 cm, preferably between 170 cm to 180 cm.

In its linking state, the detachable connecting element can connect a front-end portion of the first packaging part and a front-end portion of the second packaging part. Thus, the front-end portion of the first packaging part and the front-end portion of the second packaging part face each other when the detachable connecting element is present in its linking state. Expressed in other words, the detachable connecting element serves, in its linking position, for a front-end facing connection between the first packaging part and the second packaging part.

When the detachable connecting element is present in its non-linking state, a restriction free relative movement between the first packaging element and the second connecting element is enabled. Thus, in its non-linking state, the detachable connecting element does not impart the free movement between the first packaging part and the second packaging part.

By providing the aforementioned detachable connecting element, the overall possibilities of arranging the packaging parts with respect to each other is significantly increased. Therefore, a medical doctor or other user of a medical device contained within the packaging arrangement has more flexibility to arrange the individual packaging parts according to his or her needs. At the same time, the detachable connecting element serves, as long as it is in its linking state, for secure connection between the first packaging part and the second packaging part. Thus, as long as the user has not actively detached the detachable connecting element so as to transfer it into its non-linking state, the packaging arrangement serves for secure storage of the medical device contained therein.

In an embodiment, the medical device to be contained by the packaging arrangement is a catheter, in particular a delivery catheter for an implant, in particular a delivery catheter for delivering an implant to an implantation site through a blood vessel of a patient. In an embodiment, the implant is a heart valve prosthesis, e.g., an aortic valve prosthesis. In an embodiment, the delivery catheter is intended to be used in TAVI or TAVR applications.

The packaging arrangement can include at least one rinsing tray. In an embodiment, the detachable connecting rinsing tray can be a one-piece element. In this embodiment, the detachable connecting element in its linking state contacts the first packaging part and the second packaging part. In contrast, in its non-linking state, the detachable connecting element is removed from at least one of the first packaging part and the second packaging part so as to allow a free relative movement between the first packaging part and the second packaging part.

It is another object to provide a kit including the packaging arrangement with the aforementioned detachable connecting element and a medical device being housed within the packaging arrangement. The medical device in such a kit can be a catheter, in particular a delivery catheter for an implant, in particular a delivery catheter for delivering an implant to an implantation site through a blood vessel of a patient. In an embodiment, the implant is a heart valve prosthesis, e.g., an aortic valve prosthesis and the delivery catheter is intended to be used in TAVI or TAVR applications.

In an embodiment, one of the first connecting element part and the second connecting element part includes a wedge-like shaped lifting element. The other of the first connecting element part and the second connecting element part includes, in this embodiment, an interaction pin operatively coupled to the lifting element. In this context, the lifting element and the interaction pin are designed such that a movement of the first connecting element part relative to the second connecting element part around the connecting element axis causes a movement of the interaction pin along a surface of the lifting element. Such a movement of the interaction pin along the surface of the lifting element forces a movement of the first connecting element part relative to the second connecting element part along the connecting element axis. The rotational movement of the first connecting element part with respect to the second connecting element part is transferred into a relative translational movement between both connecting element parts so as to allow a disengagement of both connecting element parts.

In one embodiment a detachable connecting element is disclosed that can be present in a linking state and in a non-linking state, wherein the detachable connecting element connects a first packaging part and a second packaging part when being present in its linking state and wherein the detachable connecting element enables a (restriction-free) relative movement between a first packaging part and a second packaging part when being present in its non-linking state, wherein the detachable connecting element includes a first connecting element part and a second connecting element part, wherein the first connecting element part and the second connecting element part are relatively movable to each other around a connecting element axis, wherein one of the first connecting element part and the second connecting element part includes a wedge-like shaped lifting element and the other of the first connecting element part and the second connecting element part includes an interaction pin operatively coupled to the lifting element wherein the lifting element and the interaction pin are designed such that a movement of the first connecting element part relative to the second connecting element part around the connecting element axis causes a movement of the interaction pin along a surface of the lifting element, wherein such a movement of the interaction pin along a surface of the lifting element forces a movement of the first connecting element part relative to the second connecting element part along the connecting element axis.

The usage of the described connecting element is a particular appropriate possibility for enabling a detachment of a first packaging part from a second packaging part of a packaging for a medical device. Due to the interaction between the wedge-like shaped lifting element and the interaction pin, it is possible to allow a soft detachment of the first connecting element part from the second connecting element part since a rotational movement (requiring only small forces) results in a smooth axial detachment of both connecting element parts from each other. Thus, the connecting element enables a detachment of a first packaging part from a second packaging part without undesired concussions of a medical device contained in a packaging equipped with the described connecting element. This efficiently prevents or at least significantly reduces the risk of damages of a medical device contained in such a packaging.

In an embodiment, the interaction pin also has a wedge-like shape. Such a design of the interaction pin facilitates an interaction with the lifting element so as to allow a particularly simple engagement and disengagement of the first connecting element part and the second connecting element part.

In an embodiment, the interaction pin is essentially identical in construction to the lifting element. Then, two (almost) identically constructed components serve for arranging an engagement or disengagement of both connecting element parts.

In an embodiment, the lifting element and the interaction pin are designed such that they allow a movement of the first connecting element part relative to the second connecting element part around the connecting element axis only in a first direction for achieving a disengagement between the first packaging part and the second packaging part and only in a second direction being opposite to the first direction for achieving an engagement of the first packaging part and the second packaging part.

In another embodiment, the lifting element and the interaction pin are designed such that they allow a movement of the first connecting element part relative to the second connecting element part around the connecting element axis in two opposite directions for achieving a disengagement of the first packaging part and the second packaging part. In this embodiment, it is also possible to move the first connecting element part relative to the second connecting element part in two opposite directions in order to achieve an engagement of both connecting element parts. Such engagement results in an engagement of the first packaging part and the second packaging part.

In an embodiment, at least one of the first connecting element part and the second connecting element part includes a male connector. In this context, the other of the first connecting element part and the second connecting element part includes a female connector. Then, a relative movement of the first connecting element part and the second connecting element part in a first direction along the connecting element axis results in an engagement of the male connector and the female connector. Typically, this first direction along the connecting element axis is a direction of the first connecting element part and the second connecting element part towards each other. In this context, a relative movement of the first connecting element part and the second connecting element part in a second direction along the connecting element axis opposite to the first direction results in a disengagement of the male connector and the female connector. Typically, this second direction is a direction of the first connecting element part and the second connecting element part away from each other.

In an embodiment, the first connecting element part and the second connecting element part are held together in the linking state of the connecting element by force fit. In an embodiment, both connecting element parts are held together in the linking state of the connecting element exclusively by force fit.

In an embodiment, the packaging arrangement includes a locking element in addition to the connecting element. In this context, the locking element can be present in a locking state and in a non-locking state. In its locking state, the locking element serves at least for locking the first packaging part and the second packaging part in an arrangement in which the detachable connecting element is in its linking state.

In an embodiment, it is necessary to first remove the locking element prior to be able to move the first packaging part relative to the second packaging part or to move the first connecting element part relative to the second connecting element part. Alternatively, the same effect can be achieved, in an embodiment, if the locking element is transferred from its blocking state to its non-locking state.

In an embodiment, the locking element is constructed like the detachable connecting element according to the first aspect of the present invention. To be more precise, the locking element is, in an embodiment, a one-piece element. In this embodiment, the locking element is, in its locking state, arranged between the first packaging part and the second packaging part and contacts the first packaging part and the second packaging part. In contrast, in its non-locking state, the locking element is removed from at least one of the first packaging part and the second packaging part.

In an embodiment, the locking element cannot be only arranged between the first packaging part and the second packaging part in a single locking state, but rather in a first locking state and in a second locking state. In the first locking state, the locking element serves for locking the first packaging part and the second packaging part one behind the other in a series arrangement. Then, the locking element contacts the front-end portion of the first packaging part and the front-end portion of the second packaging part. In this arrangement, the packaging arrangement has a length that equals approximately the sum of the lengths of the first packaging part and the second packaging part. In its second locking state, the locking element serves for locking the first packaging part and the second packaging part one besides the other in a side by side arrangement. Then, the overall length of the packaging arrangement corresponds to the length of the first packaging part or the second packaging part, whichever of both is longer. Furthermore, a width of the packaging arrangement equals in this arrangement approximately the sum of the widths of the first packaging part and the second packaging part plus the width of the locking element. For enabling a larger bending radius the width of the packaging arrangement can be larger than the sum of the widths of the first packaging part and the second packaging part plus the width of the locking element.

In an embodiment, the locking element can be a detachable connecting rinsing tray including at least one pot for receiving a rinsing solution (such as a saline solution) for a medical device housed in the packaging arrangement or for a medical device that is intended to be connected to medical device housed in the packaging arrangement. Then, it is particularly easy to rinse the respective medical device within the packaging arrangement. Furthermore, this embodiment enables a particularly space-saving design of the packaging arrangement and combines two technical features in the locking element, namely a connecting feature on the one hand and the provision of a rinsing solution receptacle on the other hand.

In an embodiment, the detachable connecting rinsing tray cannot be arranged only between the first packaging part and the second packaging part in a single connecting state, but rather in a first connecting state and in a second connecting state. In the first connecting state, the detachable connecting rinsing tray serves for a series arrangement of the first packaging part and the second packaging part one behind the other. Then, the detachable connecting rinsing tray contacts (the front-end portion of) the first packaging part and (the front-end portion of) the second packaging part. In this first connecting state, the packaging arrangement has a length that equals approximately the sum of the lengths of the first packaging part and the second packaging part. Here approximately means that an overlapping portion of the first packaging part and the second packaging part has to be deducted from the sum of the lengths of the first packaging part and the second packaging part. In its second connecting state, the detachable connecting rinsing tray serves for a side by side arrangement of the first packaging part and the second packaging part one besides the other. Then, the overall length of the packaging arrangement corresponds to the length of the first packaging part or the second packaging part, whichever of both is longer. Furthermore, a width of the packaging arrangement equals in this arrangement approximately the sum of the widths of the first packaging part and the second packaging part. Approximately means that either an overlapping portion of the first packaging part and the second packaging part has to be deducted from the sum of the widths of the first packaging part and the second packaging part or a space between the first packaging part and the second packaging part has to be added to the sum of the widths of the first packaging part and the second packaging part.

In an embodiment, the detachable connecting rinsing tray includes at least one pot for storing and/or receiving a liquid, preferably a rinsing solution (such as a saline solution) for a medical device housed in the packaging arrangement or for a medical device that is intended to be connected to medical device housed in the packaging arrangement. Then, it is particularly easy to rinse the respective medical device within the packaging arrangement. Furthermore, this embodiment enables a particularly space-saving design of the packaging arrangement and combines two technical features in the detachable connecting element, namely a connecting feature on the one hand and the provision of a rinsing solution receptacle on the other hand.

In an embodiment, the detachable rinsing tray is designed and arranged such that it is accessible for a user from a top and/or from a side of the packaging arrangement when the detachable connecting element is to be transferred from its linking state to its non-linking state.

In an embodiment, the locking element is designed and arranged such that it is accessible for a user from a top and/or from a side of the packaging arrangement when the locking element is to be transferred from its linking state to its non-linking state.

The locking element is particularly provided in case that the detachable connecting element is not designed as a one-piece element, but rather as having a first connecting element part and a second connecting element part.

An aspect of the present invention relates to a connecting element for a packaging arrangement for a medical device. The connecting element is a detachable connecting element that can be present in a linking state and in a non-linking state. In its linking state, the detachable connecting element connects a first packaging part and a second packaging part. If the detachable connecting element is, however, present in its non-linking state, it enables a restriction-free relative movement between a first packaging part and a second packaging part. The detachable connecting element includes a first connecting element part and a second connecting element part. The first connecting element part and the second connecting element part are relatively movable to each other around a connecting element axis. One of the first connecting element part and the second connecting element part includes a wedge-like shaped lifting element. The other of the first connecting element part and the second connecting element part includes an interaction pin operatively coupled to the lifting element. In this context, the lifting element and the interaction pin are designed such that a movement of the first connecting element part relative to the second connecting element part around the connecting element axis causes a movement of the interaction pin along a surface of the lifting element. Such a movement of the interaction pin along a surface of the lifting element forces a movement of the first connecting element part relative to the second connecting element part along the connecting element axis. If this movement is a relative movement of both connecting element parts towards each other, an engagement of both connecting element parts is achieved. If the movement is a relative movement of both connecting element parts away from each other, a disengagement of both connecting element parts is achieved.

In one embodiment the packaging arrangement is characterized in that one of the first connecting element part and the second connecting element part includes a wedge-like shaped lifting element and the other of the first connecting element part and the second connecting element part includes an interaction pin operatively coupled to the lifting element, wherein the lifting element and the interaction pin are designed such that a movement of the first connecting element part relative to the second connecting element part around the connecting element axis causes a movement of the interaction pin along a surface of the lifting element, wherein such a movement of the interaction pin along a surface of the lifting element forces a movement of the first connecting element part relative to the second connecting element part along the connecting element axis. The interaction pin is essentially complementary in construction to the lifting element. The lifting element and the interaction pin can be designed such that they allow a movement of the first connecting element part relative to the second connecting element part around the connecting element axis only in a first direction for achieving a disengagement of the first packaging part and the second packaging part. Alternatively the lifting element and the interaction pin can be designed such that they allow a movement of the first connecting element part relative to the second connecting element part around the connecting element axis in two opposite directions for achieving a disengagement of the first packaging part and the second packaging part.

All embodiments and variants of the packaging arrangement can be combined in any desired way and can be transferred in any desired combination to the connecting element.

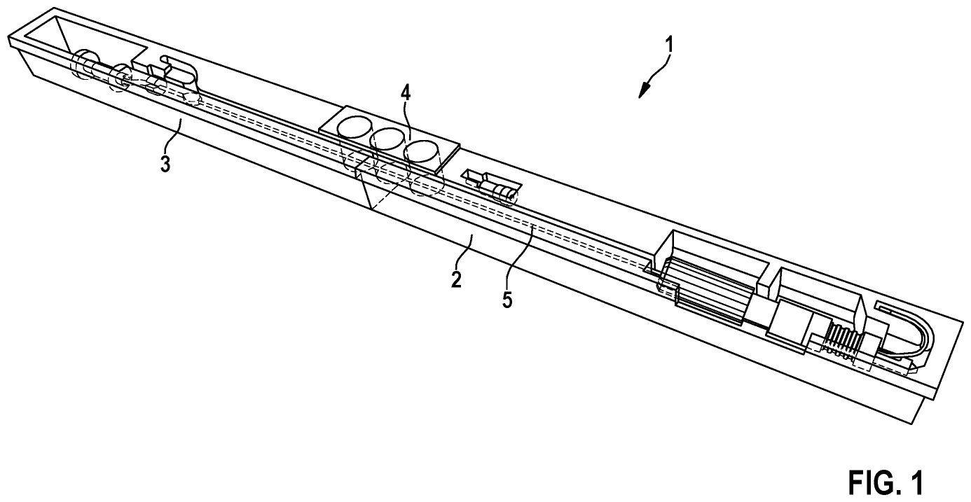

FIG. 1 shows a TAVI/TAVR delivery catheter packaging that serves as packaging arrangement 1 for a medical device. The packaging arrangement 1 includes a first packaging part 2, preferably a handle tray and a loading basin tray that serves as second packaging part 3. The handle tray and the loading basin tray are connected to each other by a detachable connecting rinsing tray 4.

The packaging arrangement 1 is a (thermoformed) blister tray and can be manufactured from various plastic materials, preferably a plastic material for medical products. The TAVI/TAVR delivery catheter packaging arrangement 1 serves for housing a TAVI/TAVR delivery catheter 5 and is provided with recesses so as to be specifically adapted to house the TAVI/TAVR delivery catheter 5. The TAVI/TAVR delivery catheter 5 is an oblong medical device that serves for delivering an aortic valve implant to the aorta of a patient in need thereof.

The TAVI/TAVR delivery catheter packaging arrangement 1 shown in FIG. 1 is a transport or storage state. In this transport or storage state, the (oblong) handle tray 2 and the (oblong) loading basin tray 3 are connected to each other, preferably at their front-end portions, by the connecting rinsing tray 4. Preferably, the TAVI/TAVR delivery catheter packaging arrangement 1 has an overall oblong appearance in its transport or storage state, wherein its length corresponds (approximately) to the sum of the length of the handle tray 2 and the loading basin tray 3

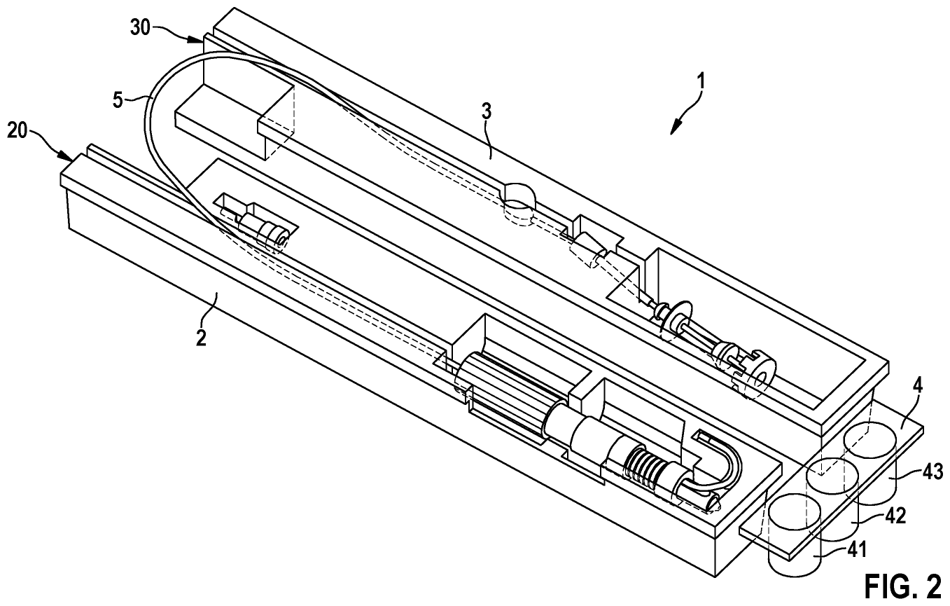

When the TAVI/TAVR delivery catheter 5 contained in the TAVI/TAVR delivery catheter packaging arrangement 1 is to be used by a surgeon in order to implant an aortic valve implant to a patient, the TAVI/TAVR delivery catheter packaging arrangement 1 is opened and is transferred to its preparation state, cf. FIG. 2. Here and in all following Figures, the same or similar elements will be denoted with the same numeral references.

To transfer the TAVI/TAVR delivery catheter packaging arrangement 1 from its storage state to its preparation state, the connecting rinsing tray 4 is removed from the handle tray 2 and from the loading basin tray 3. The connecting rinsing tray 4 may then be neither connected to the handle tray 2 nor to the loading basin tray 3 in the depiction of FIG. 2. Then, the handle tray 2 and the loading basin tray 3 can be generally moved with respect to each other. In the embodiment shown in FIG. 2, the relative movement between the handle tray 2 and the loading basin tray 3 is restricted by the TAVI delivery catheter 5 which extends from the handle tray 2 towards the loading basin tray 3.

However, in the preparation state of the TAVI/TAVR delivery catheter packaging arrangement 1, the connecting rinsing tray 4 is typically connected to at least one of the handle tray 2 and the loading basin tray 3 (often, it is connected to both of the handle tray 2 and the loading basin tray 3). Then, it tightly keeps the handle tray 2 and the loading basin tray 3 in place also in the preparation state of the TAVI/TAVR delivery catheter packaging arrangement 1.

The connecting rinsing tray 4 includes several rinsing sinks 41, 42, 43, preferably a first rinsing sink 41, a second rinsing sink 42, and a third rinsing sink 43, which serve as recesses for a rinsing solution and are intended to receive a rinsing solution so as to be able to rinse an implant (e.g. a stent) to be implanted by the TAVI/TAVR delivery catheter 5 into the aorta of a patient.

In the depiction of FIG. 2, a front-end portion 20 of the handle tray 2 and a front-end portion 30 of the loading basin tray 3 can be seen. These front-end portions 20, 30 are connected to each other in the transport or storage state of the TAVI/TAVR delivery catheter packaging arrangement 1, as shown in FIG. 1.

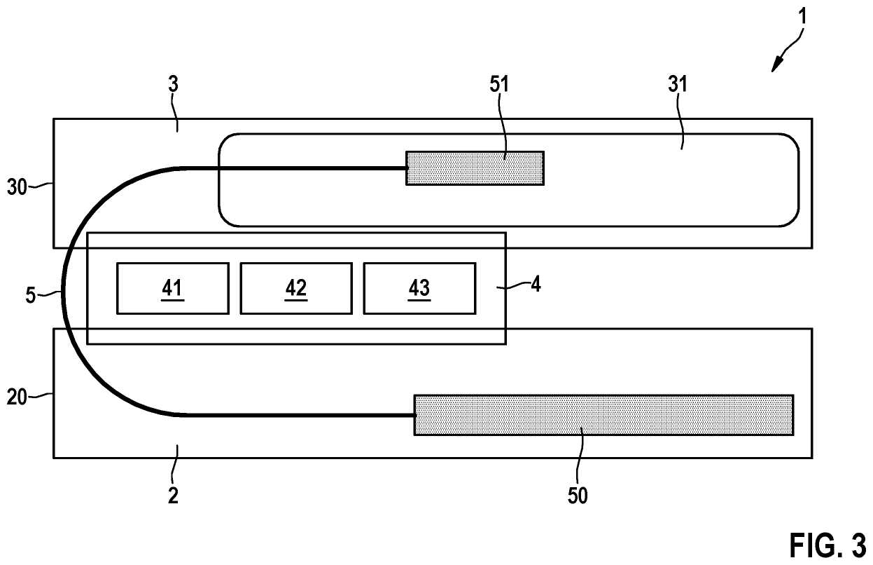

FIG. 3 shows a schematic depiction of the TAVI/TAVR delivery catheter packaging arrangement 1 of FIGS. 1 and 2 from the top. Here, a possible position of the connecting rinsing tray 4 is shown. In this context, the connecting rinsing tray 4 is connected to a lateral side of the handle tray 2 and a lateral side of the loading basin tray 3 so as to enable an arrangement of the handle tray 2 and the loading basin tray 3 beside each other. In this arrangement, it is particularly easy to access the first rinsing sink 41, the second rinsing sink 42 and the third rinsing sink 43 of the connecting rinsing tray 4 to allow for rinsing of an implant to be implanted with the help of the TAVI/TAVR delivery catheter 5.

A handle 50 of the TAVI/TAVR delivery catheter 5 is arranged within the handle tray 2. A loading portion 51 of the TAVI/TAVR delivery catheter 5 is arranged within a loading basin 31 of the loading basin tray 3.

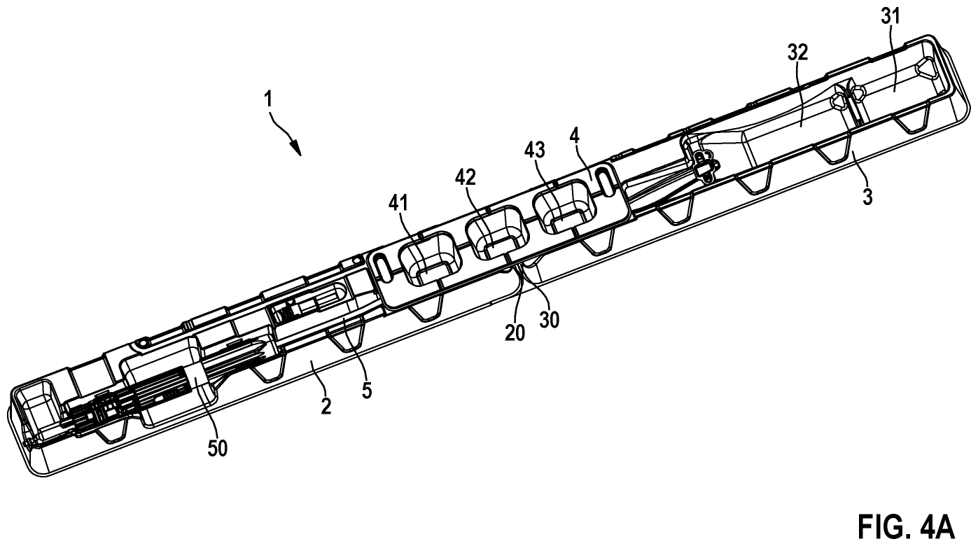

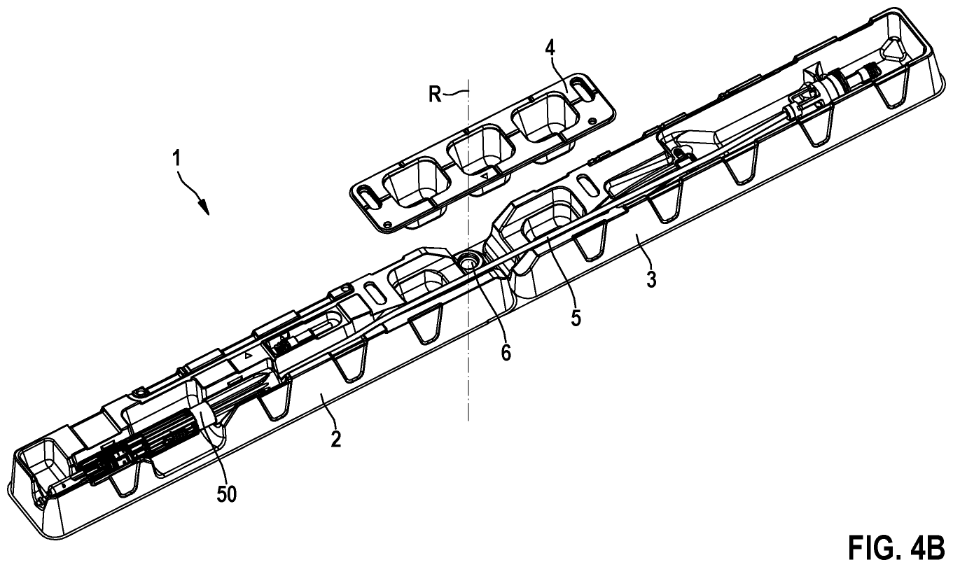

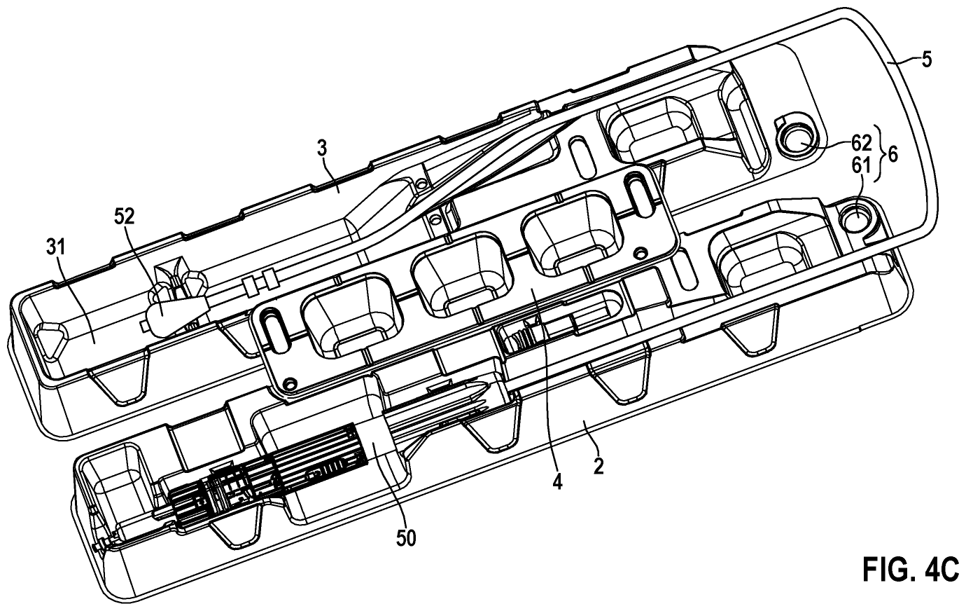

FIG. 4A shows another embodiment of a TAVI/TAVR delivery catheter packaging arrangement 1 containing a TAVI/TAVR delivery catheter 5. In the depiction of FIG. 4A, the loading portion of the TAVI/TAVR delivery catheter 5 is not depicted in detail so as to allow a better view into the loading basin 31 of the loading basin tray 3. Here, an optional mirror 32 can be seen which is attached to the bottom of the loading basin 31. This mirror facilitates loading of an aortic valve implant into the loading portion of the TAVI/TAVR delivery catheter 5.

A unique identifier (for example in the form of a simple digit) can be embossed into the bottom of the first rinsing sink 41, the second rinsing sink 42 and the third rinsing sink 43 of the connecting rinsing tray 4 so as to allow an unique identification of each of the rinsing sinks 41, 42, 43.

In the embodiment depicted in FIG. 4A, the connecting rinsing tray 4 does not only serve as a further detachable connecting element, but also as a locking element. This will be apparent when turning to FIGS. 4B and 4C.

It can be seen in FIG. 4B that the connecting rinsing tray 4 is already removed from the handle tray 2 and the loading basin tray 3. However, it is still not possible to freely move the handle tray 2 and the loading basin tray 3 with respect to each other since a ring-shaped connecting element 6 still serves for a tight connection between the handle tray 2 and the loading basin tray 3. Thus, this ring-shaped connecting element 6 serves as detachable connecting element in this embodiment.

It is possible to rotate the loading basin tray 3 around a rotational axis R serving as connecting element axis. By such a rotation, disengagement of two parts of the ring-shaped connecting element 6 will occur. This is depicted in FIG. 4C.

FIG. 4C shows the TAVI/TAVR delivery catheter packaging arrangement 1 in its preparation state. In this state, a first connecting element part 61 can be seen on the bottom of the handle tray 2. Likewise, a second connecting element part 62 can be seen on the bottom of the loading basin tray 3. Both connecting element parts 61 and 62 from together the connecting element 6 when engaging each other. The second connecting element part 62 includes a ring-shaped protrusion and the first connecting element part 61 includes a ring-shaped recess for incorporating the ring-shaped protrusion of the second connecting element part 62.

If the first ring-shaped connecting element part 61 and the second ring-shaped connecting element part 62 are disengaged (like in the depiction of FIG. 4C), a free relative movement of the handle tray 2 and the loading basin tray 3 is generally possible. In the preparation state depicted in FIG. 4C, the connecting rinsing tray 4 is already arranged between the handle tray 2 and the loading basin tray 3 and locks both trays with respect to each other so that their relative movement is restricted. The connecting rinsing tray is positioned in a recessed area, which is partially formed by the handle tray 2 and the loading basin tray 3. This allows an easy preparation of the TAVI/TAVR delivery catheter 5 contained within the TAVI/TAVR delivery catheter packaging arrangement 1. A medical doctor can then load the TAVI/TAVR delivery catheter 5 with an aortic valve implant 52 in the loading basin 31. For this purpose, the loading basin 31 is typically filled with an ice-cold saline solution.

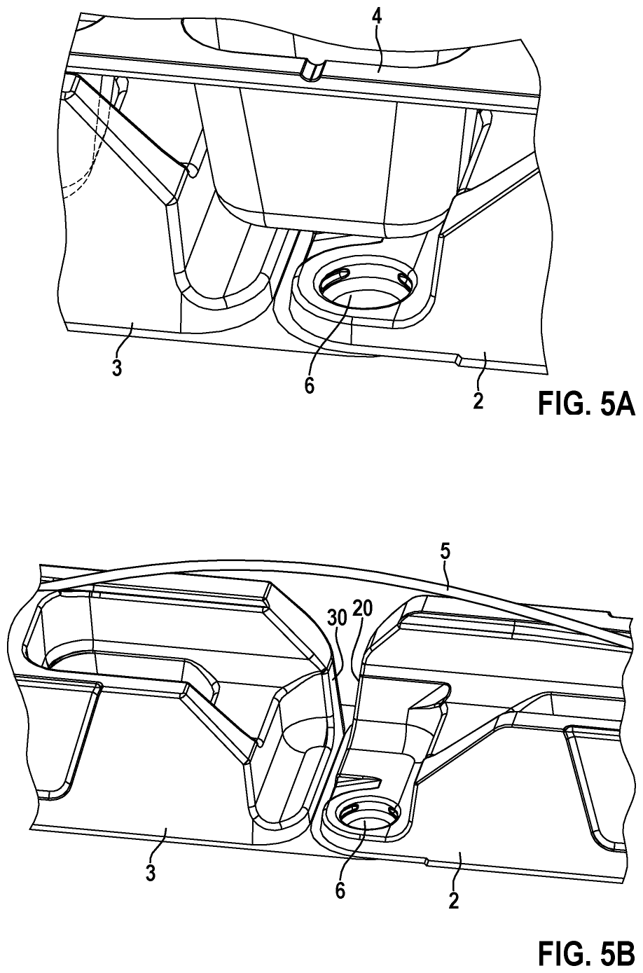

FIG. 5A shows a detail of a connection portion of the TAVI/TAVR delivery catheter packaging arrangement 1 shown in FIGS. 4A to 4C. Here, it can well be seen that the TAVI/TAVR delivery catheter packaging arrangement 1 includes both the ring-shaped connecting element 6 serving as detachable connecting element as well as the connecting rinsing tray 4 serving as additional locking element. In the depiction of FIG. 5A, the connecting rinsing tray 4 is in its connecting state in which it connects a front-end side of the handle tray 2 and a front-end side of the loading basin tray 3 so that the handle tray 2 and the loading basin tray 3 are arranged one behind the other. The recesses and/or protrusions sidewall has an indentation/and or protuberance (being oriented perpendicular to the rotation axis).

In the depiction of FIG. 5B, the connecting rinsing tray 4 is removed from the TAVI/TAVR delivery catheter packaging arrangement 1. Still then, the handle tray 2 and the loading basin tray 3 are connected to each other via the ring-shaped connecting element 6. The recesses and/or protrusions sidewall has an indentation/and or protuberance (being oriented perpendicular to the rotation axis).

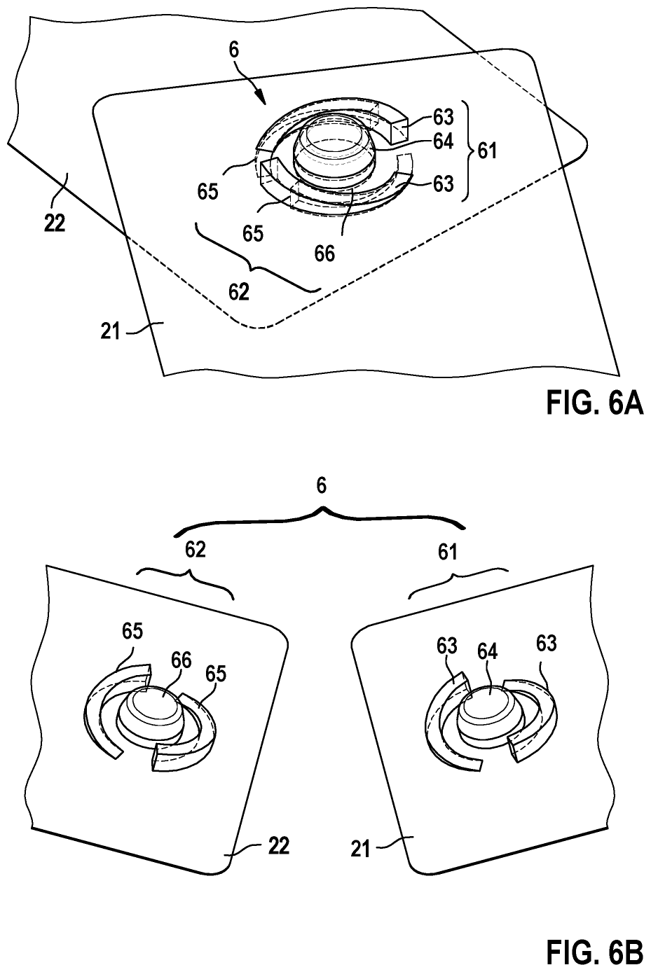

FIG. 6A shows a first embodiment of a connecting element 6 including two connecting element parts 61, 62 in a linking state, of which a first connecting element part 61 is represented in the figure by a solid line and the second element part 62 is represented in the figure by a dashed line. This first connecting element part 61 includes a base body 21 and (at least) two wedge-shaped ramps 63 and a central connecting element 64. Thereby, the ramps 63 and the central connecting element 64 are embossed into the base body 21 of the first connecting element part 61, e.g. a handle tray of a TAVI/TAVR delivery catheter packaging. The second connecting element part 62 includes a base body 22 and (at least) two wedge-shaped ramps 65 and a central connecting element 66. Thereby, the ramps 65 and the central connecting element 66 are embossed into the base body 22 of second connecting element part 62, e.g. a loading basin tray of a TAVI/TAVR delivery catheter packaging.

Similar elements with slightly smaller dimensions are embossed into the base body of the second connecting element part 62, e.g. a loading basin tray of the same TAVI/TAVR delivery catheter packaging. If the base body 21 of the first connecting element part 61 (e.g. of the handle tray) is then placed above the base body 22 of the second connecting element part 62 (e.g. of the loading tray) the wedge-shaped ramps of second connecting element part 62 can engage an interior part of the wedge-shaped ramps 63 of the first connecting element part 62. Likewise, a central connecting element 66 of the second connecting element part 62 (e.g. positioned on the loading basin tray) can engage an interior of the central connecting element 64 of the first connecting element part (e.g. positioned on the handle tray). Then, a tight connection between the first connecting element part 61 (e.g. positioned on the handle tray) and the second connecting element part 62 (e.g. positioned on the loading basin tray) can be achieved by simply rotating the first connecting element part 61 (or e.g. the handle tray) against the second connecting element part 62 (or e.g. loading basin tray). However, the first connecting element part 61 and the second connecting element part 62 can also only be pressed onto each other to achieve a form fit/and/or force fit connection.

FIG. 6B shows the first connecting element part 61 and the second connecting element part 62 in a non-linking state. Here, the three recesses 63, 64, namely two wedge-shaped ramps 63 bent around the rotation axis and a hemispherical shaped central connecting element 64, protrude from the base body 21 of the first connecting element part 61 can be seen as in FIG. 6A. Additionally, the second connecting element part 62 with three protrusions 65, 66, namely two wedge-shaped ramps 65 bent around the rotation axis and a central hemispherical shaped connecting element 66 protruding from the base body of the second connecting element part 62 is visible. As explained with respect FIG. 6A, the bent wedge-shaped ramps of the second connecting element part 62 can engage an interior hollow space of the bent wedge-shaped ramps of the first connecting element part 61. Likewise, the central hemispherical shaped connecting element 66 of the second connecting element part 62 can engage an interior hollow space of the central hemispherical shaped connecting element of the first connecting element part 61. The detachable connecting element 6 can be locked by rotating the first connecting element part 61 or the second connecting element part 62 in one direction around the rotation axis R and the detachable connecting element 6 can be unlocked by rotating the first connecting element part 61 or the second connecting element part 62 in a direction opposite to the one direction.

In this context, the hollow space of the central connecting element 64 of the first connecting element part 61 serves as female connecting element, whereas the central connecting element 66 of the second connecting element 62 acts as male connector. The male and female connector can be connected by a form fit and/or force fit connection.

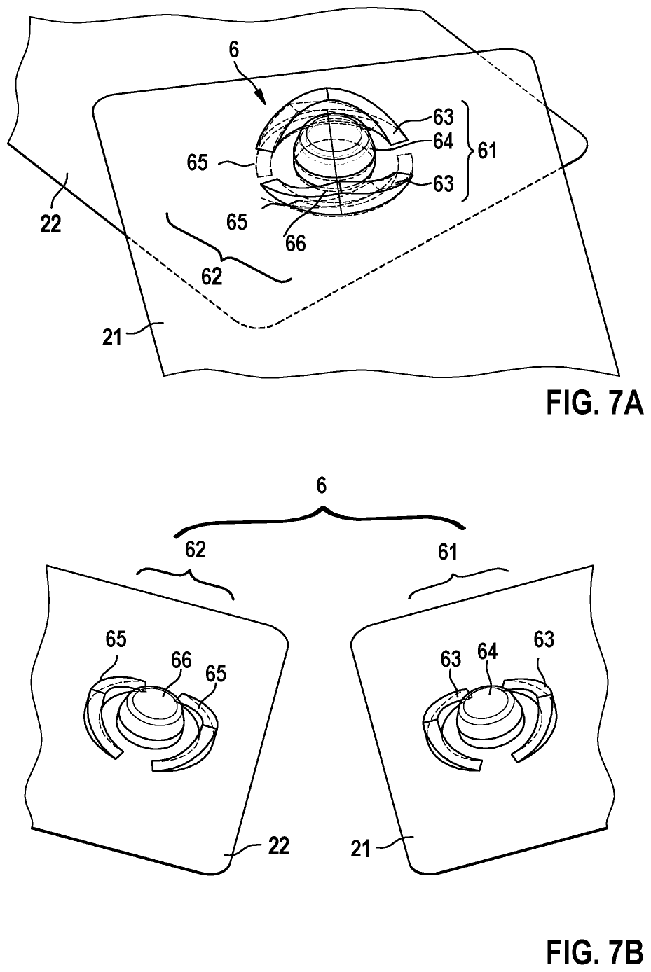

FIG. 7A shows a similar embodiment of a connecting element 6 like FIG. 6A. The first connecting element part 61 includes a base body 21 and three recesses 63, 64, namely two trigonal prisms 63 bent around the rotation axis and a central hemispherical shaped connecting element 64. The second connecting element part 62 includes a base body 22 and three protrusion 63, 64, namely two trigonal prisms 63 bent around the rotation axis and a central hemispherical shaped connecting element 64. In this context, the bent wedge-shaped ramps 63 are designed such that a relative movement between the first connecting element part 61 (e.g., provided on a handle tray of a packaging for a medical device) and a second connecting element part (e.g., provided on a loading basin tray of the packaging for a medical device) enables an engagement or disengagement, respectively, of the connecting element parts 61, 63 or of the base body 21 of the first connecting element part 61 (e.g. the handle tray) and the base body of the second connecting element part 62 (e.g. the loading basin tray), respectively.

FIG. 7B shows the first connecting element part 61 and the second connecting element 62 including each two recesses and protrusions having a bent trigonal prism shape. A rotation in either direction serves for engagement, wherein further rotation in the same direction results in the disengagement of the individual parts. In this context, the central connecting element 66 of the second connecting element part 62 still enters a hollow space of the central connecting element 64 of the first connecting element part 61 for establishing an engagement between the first connecting element part 61 and the second connecting element part 62. Likewise, the central connecting element 66 of the second connecting element part 62 leaves the hollow space of the central connecting element 64 of the first connecting element part 61 for establishing a disengagement between the first connecting element part 61 and the second connecting element part 62.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A detachable connecting element for a packaging arrangement, comprising:
a first connecting element part and a second connecting element part configured for relative movement with respect to each other around a connecting element axis (R) by a rotational or helical movement to transfer the detachable connecting element from a linking state to a non-linking state, wherein:
the second connecting element part comprises at least one protrusion;
the first connecting element part comprises at least one recess configured to incorporate the at least one protrusion;
the at least one protrusion comprises at least one curved protrusion sidewall;
the at least one recess comprises at least one curved recess sidewall; and
the at least one curved protrusion sidewall or the at least one curved recess sidewall comprises a slope.

2. The detachable connecting element according to claim 1, wherein the first connecting element part and the second connecting element part are configured such that the relative movement in a first direction results in an engagement of the first element part and the second element part and the relative movement in a second direction opposite to the first direction results in a disengagement of the of the first element part and the second element part.

3. The detachable connecting element according to claim 1, wherein the at least one curved protrusion sidewall comprises a slope, preferably wherein the slope of the at least one protrusion sidewall and/or the at least one curved protrusion sidewall has an angle of inclination of between 1 degree and 60 degrees or between 20 degrees and 45 degrees.

4. The detachable connecting element according to claim 1, wherein the at least one protrusion is a male connector and the at least one recess is a female connector.

5. The detachable connecting element according to claim 4, wherein the at least one female connector and the at least one male connector comprise a hemispherical shape, a cylindrical shape, a dome shape, a conical shape, a cone shape or a truncated cone shape.

6. The detachable connecting element according to claim 1, wherein one of the at least one recess and the at least one protrusion comprise a bent triangular shaped sidewall.

7. The detachable connecting element according to claim 1, wherein the at least one protrusion and/or the at least one recess comprises a bent wedge shape or a bent trigonal prism shape.

8. The detachable connecting element according to claim 1, wherein the first connecting element part and the second connecting element part are configured to create a force fit and/or form fit in the linking state.

9. A packaging arrangement for a medical device comprising the detachable connecting element of claim 1, the packaging arrangement comprising a first packaging part, a second packaging part arranged with the detachable connecting element to be connectable into connecting state.

10. The packaging arrangement according to claim 9, comprising at least one detachable connecting rinsing tray configured to store and/or receive a liquid.

11. The packaging arrangement according to claim 10, wherein the at least one detachable connecting rinsing tray is a one-piece element that contacts the first packaging part and the second packaging part in the connecting state and is configured to be removed from the at least one of the first packaging part and the second packing part in a non-connecting state.

12. The packaging arrangement according to claim 10, wherein the detachable connecting rinsing tray is configured to be arranged between the first packaging part and the second packaging part in a first connecting state and in a second connecting state.

13. The packaging arrangement according to claim 10, wherein the detachable connecting rinsing tray is configured to provide a series arrangement of the first packaging part and the second packaging part one behind the other in the first connecting state, and configured to provide a side by side arrangement of the first packaging part and the second packaging part in the second connecting state.

14. The packaging arrangement according to claim 10, wherein the detachable connecting rinsing tray comprises at least one rinsing sink configured to receive a solution.

15. The packaging arrangement according to claim 14, wherein the first packaging part and/or the second packaging part together form at least one recessed area for receiving the at least one rinsing sink.

16. The detachable connecting element according to claim 1, wherein the slope has an angle of inclination of between 1 degree and 60 degrees.

17. The detachable connecting element according to claim 16, wherein the slope has an angle of inclination or between 20 degrees and 45 degrees.
